(19) United States
(12) Patent Application Publication
(10) Pub. No.: US 2026/0283656 A1
(43) Pub. Date: Sep. 24, 2026

(54) SCREW IMPLANTATION DEVICE

(71) Applicant: Point Robotics Medtech Inc., Hsinchu County (TW)

(72) Inventor: Ren-Jeng Wang, Hsinchu County (TW)

(21) Appl. No.: 19/565,559

(22) Filed: Mar. 13, 2026

Related U.S. Application Data
(63) Continuation-in-part of application No. 19/300,628, filed on Aug. 14, 2025.

(30) Foreign Application Priority Data
Mar. 21, 2025 (TW) 114110660
Jan. 6, 2026 (TW) 115100353

(51) Int. Cl. A61B 17/70; A61B 90/00; A61B 17/00

(52) U.S. Cl. CPC A61B 17/7082; A61B 17/7032; A61B 90/03; A61B 2017/00398; A61B 2017/00477; A61B 2090/034

(57) ABSTRACT

A screw implantation device includes a transmission assembly and a guide pin assembly. The transmission assembly includes a hollow transmission shaft and a transmission connecting member. The hollow transmission shaft has a first end and a second end, and the transmission connecting member is rotatably sleeved around the first end. Two latching protrusions are disposed on two sides of the transmission connecting member. The guide pin assembly is detachably assembled to the first end. The guide pin assembly includes a socket member, a coupling member, and a guide pin. The hollow transmission shaft is sleeved around the socket member and connected to the coupling member. Two first protruding portions are disposed on two sides of the coupling member and engaged with the latching protrusions. The guide pin passes through the coupling member, and is connected to the socket member.

### CROSS-REFERENCE TO RELATED PATENT APPLICATION

This application is a continuation-in-part application of the U.S. Application Ser. No. 19/300,628, filed on Aug. 14, 2025 and entitled “SCREW IMPLANTATION DEVICE”, the contents of which are incorporated herein by reference in their entireties.

This application also claims the benefit of priorities to Taiwan Patent Application No. 114110660, filed on Mar. 21, 2025, and No. 115100353, filed on Jan. 6, 2026. The entire content of each of the above identified applications is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a screw implantation device, and more particularly to a screw implantation device for simplifying steps of pedicle screw implantation.

### BACKGROUND OF THE DISCLOSURE

Spinal fusion surgery is a common procedure for treating lower back pain, and usually involves steps of: making a small incision on the skin surface, creating a guide hole by using instruments such as a trocar or a bone drill, and then inserting a guide wire. Afterwards, a hollow pedicle screw is locked into the spine along the guide wire, so as to complete implantation and stabilization of pedicle screws.

However, a high rotational speed with a low torque is generally required during a drilling stage, whereas a low rotational speed with a high torque is required for insertion of the pedicle screws. Thus, different instruments are used in different procedures. In other words, conventional processes for implanting the pedicle screws involve complicated surgical steps and exchange of instruments, such that certain operational steps may easily be neglected by a surgeon. In addition, frequent change of instruments may sometimes result in loosening or movement of the instruments, thereby causing positioning deviation (i.e., poor drilling alignment accuracy) or causing a final implantation position to be different from that in preoperative planning.

Therefore, how to overcome the above-mentioned problems through improvements in structural design has become one of the important issues to be addressed in the related art.

### SUMMARY OF THE DISCLOSURE

In response to the above-referenced technical inadequacies, the present disclosure mainly provides a screw implantation device, so as to overcome problems of loosening or movement of instruments caused by complicated operational processes in conventional screw implantation and frequent exchange of the instruments.

In order to solve the above-mentioned problems, one of the technical aspects adopted by the present disclosure is to provide a screw implantation device, which includes a transmission assembly and a guide pin assembly. The transmission assembly includes a hollow transmission shaft and a transmission connecting member. The hollow transmission shaft has a first end and a second end, and a plurality of limiting members are disposed at the first end. The transmission connecting member is rotatably sleeved around the first end. Two latching protrusions are disposed on two sides of the transmission connecting member, and each of the latching protrusions has a dent. The guide pin assembly is detachably assembled to the first end of the hollow transmission shaft. The guide pin assembly includes a socket member, a coupling member, and a guide pin. The coupling member has a hollow structure, and is detachably connected to the socket member. The hollow transmission shaft is sleeved around the socket member, and is connected to the coupling member. Two first protruding portions are disposed on two sides of the coupling member. The guide pin passes through the coupling member, and is connected to the socket member. When the guide pin assembly is assembled to the hollow transmission shaft, the transmission connecting member is configured to rotate around an axis of the hollow transmission shaft, such that the two first protruding portions are respectively engaged in the two dents and blocked by the two latching protrusions, so as to fix the guide pin assembly in place.

Therefore, in the screw implantation device provided by the present disclosure, through the design of the first protruding portions and the latching protrusions, the guide pin assembly can be stably and reliably assembled to the hollow transmission shaft. When the guide pin assembly is assembled to the hollow transmission shaft, the transmission connecting member rotates around the axis of the hollow transmission shaft, such that the two first protruding portions are respectively engaged in the two dents and blocked by the two latching protrusions. In this way, the guide pin assembly is effectively fixed in place, and loosening or movement of the guide pin assembly can be prevented, thereby enhancing stability and operational safety of a device.

These and other aspects of the present disclosure will become apparent from the following description of the embodiment taken in conjunction with the following drawings and their captions, although variations and modifications therein may be effected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a screw implantation device according to the present disclosure;

FIG. 2 is a schematic partially exploded view of the screw implantation device according to the present disclosure;

FIG. 3 is a schematic exploded view of a transmission assembly, a guide pin assembly, and a pedicle screw assembly of the screw implantation device according to the present disclosure;

FIG. 4 is a schematic exploded view of the transmission assembly according to the present disclosure;

FIG. 5 is a schematic partial view of the transmission assembly and the guide pin assembly according to the present disclosure;

FIG. 6 is a schematic view showing a portion of the transmission assembly according to the present disclosure;

FIG. 7 is a schematic view showing another portion of the transmission assembly according to the present disclosure;

FIG. 8 is a schematic cross-sectional view of the guide pin assembly of the screw implantation device according to the present disclosure;

FIG. 9 is a schematic view showing assembly of the transmission assembly and the guide pin assembly according to the present disclosure;

FIG. 10 is another schematic view showing assembly of the transmission assembly and the guide pin assembly according to the present disclosure;

FIG. 11 is a schematic view of the pedicle screw assembly and a retaining member according to the present disclosure; and

FIG. 12 is a schematic exploded view of the pedicle screw assembly according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Embodiment

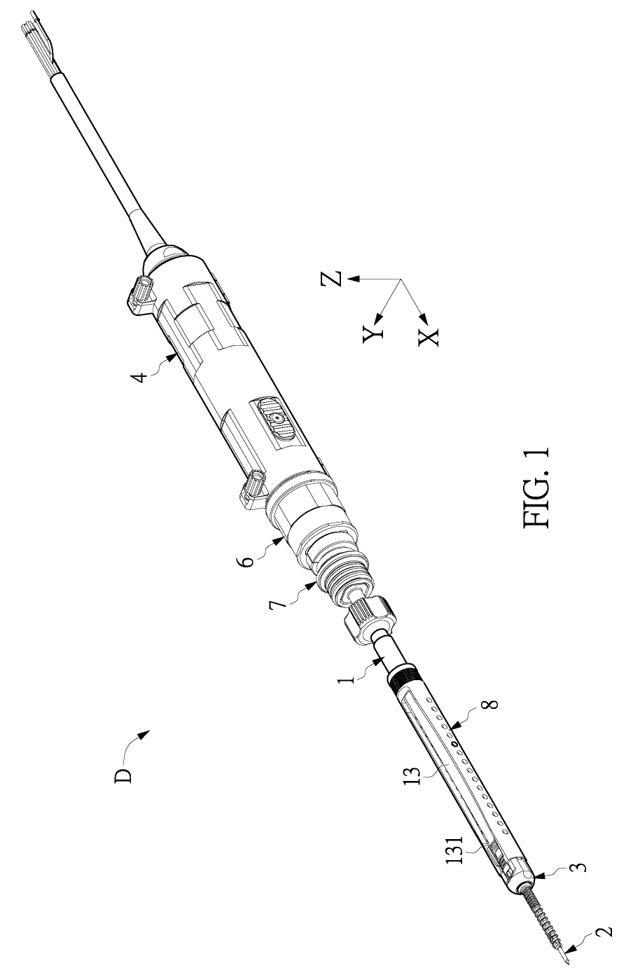

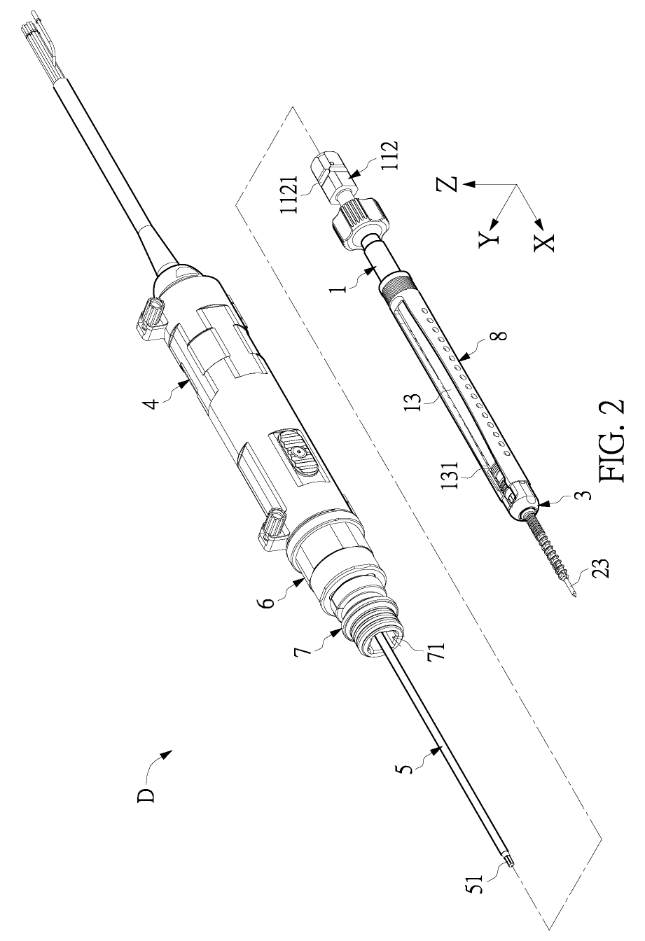

Referring to FIGS. 1 and 2, FIG. 1 is a schematic view of a screw implantation device according to the present disclosure, and FIG. 2 is a schematic partially exploded view of the screw implantation device according to the present disclosure. The present disclosure provides a screw implantation device D, which includes a transmission assembly 1, a guide pin assembly 2, a pedicle screw assembly 3, a driving motor 4, a central shaft 5, a reducer assembly 6, a quick-release connector 7, and a retaining member 8.

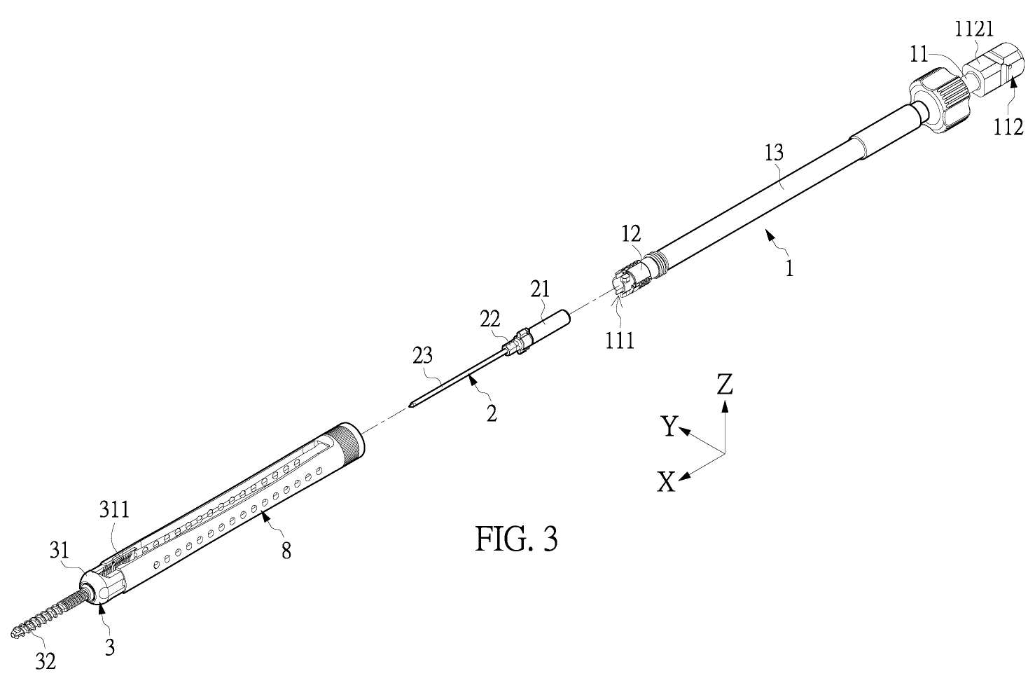

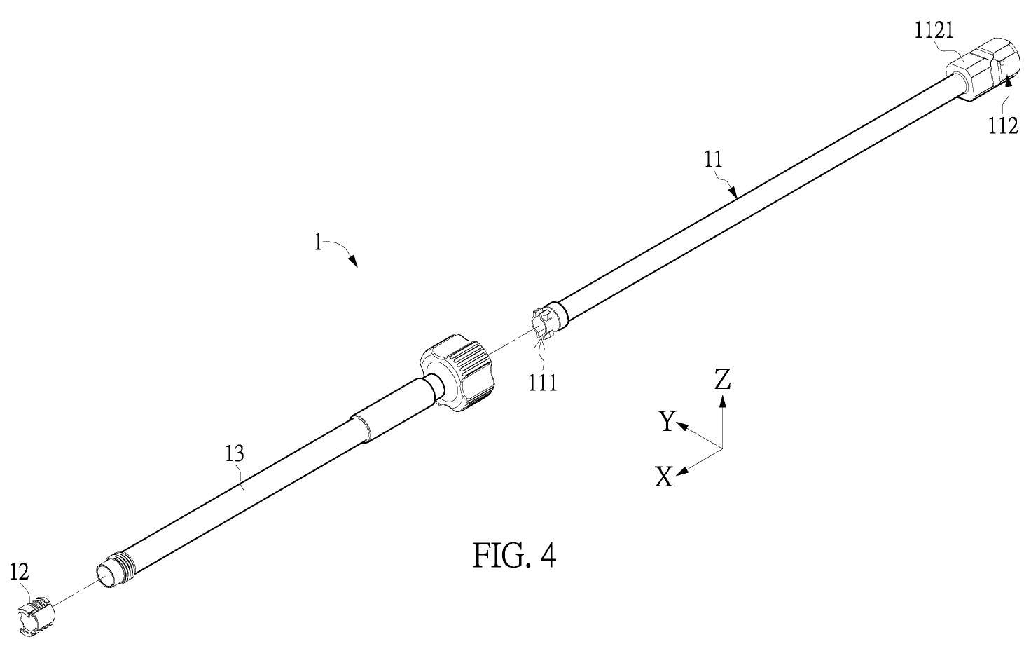

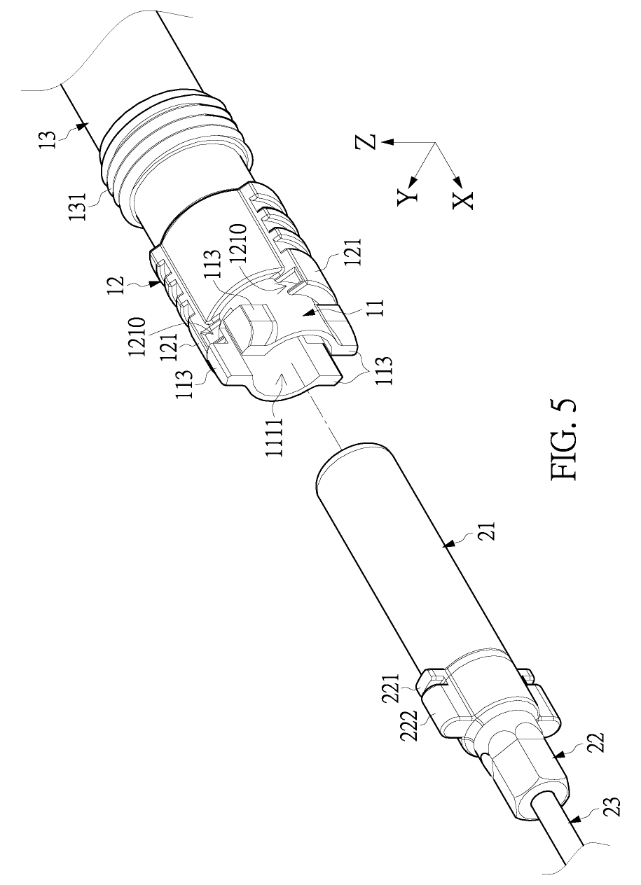

Referring to FIGS. 3-5, FIG. 3 is a schematic exploded view of a transmission assembly, a guide pin assembly, and a pedicle screw assembly of the screw implantation device according to the present disclosure, FIG. 4 is a schematic exploded view of the transmission assembly according to the present disclosure, and FIG. 5 is a schematic partial view of the transmission assembly and the guide pin assembly according to the present disclosure. The transmission assembly 1 includes a hollow transmission shaft 11, a transmission connecting member 12, and a hollow connecting shaft 13. The hollow transmission shaft 11 has a first end 111 and a second end 112. A port 1111 is formed at the first end 111 of the hollow transmission shaft 11, a plurality of limiting members 113 are disposed on an outer side surface of the port 1111, and an engaging portion 1121 is disposed at the second end 112.

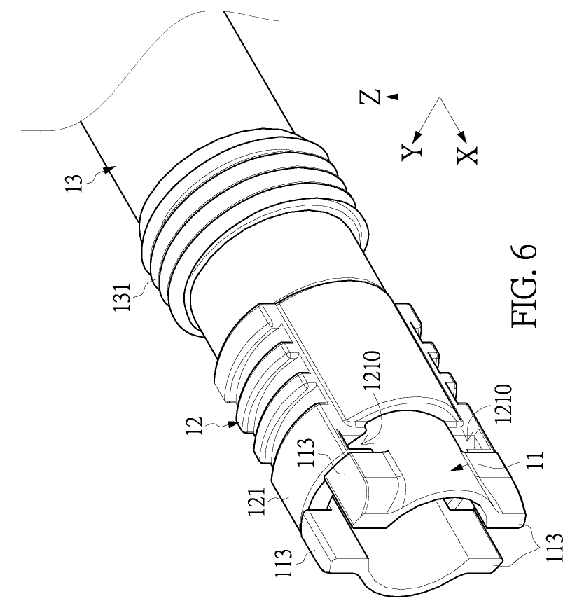

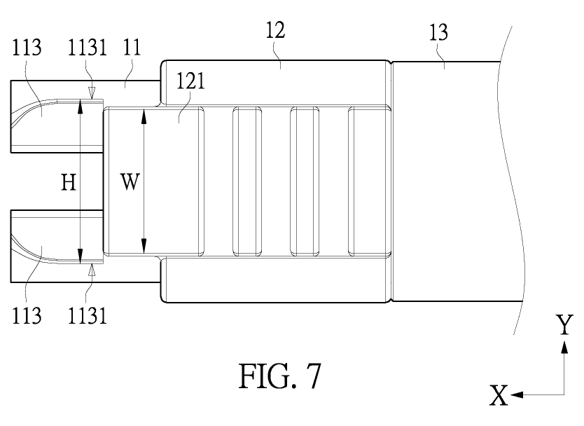

The transmission connecting member 12 and the hollow connecting shaft 13 each have a hollow structure, and are sleeved around the hollow transmission shaft 11. One end of the hollow connecting shaft 13 abuts against the transmission connecting member 12. In this way, the transmission connecting member 12 is limited at a position of the first end 111 of the hollow transmission shaft 11, and is disposed between the limiting members 113 and the hollow connecting shaft 13. Furthermore, as shown in FIGS. 6 and 7, when the transmission connecting member 12 is disposed on the hollow transmission shaft 11, the transmission connecting member 12 is in a relatively rotatable state. Two latching protrusions 121 are symmetrically disposed on two sides of the transmission connecting member 12, and each of the latching protrusions 121 has a dent 1210.

Reference is still made to FIGS. 6 and 7. In one embodiment of the present disclosure, a quantity of the limiting members 113 disposed at the first end 111 of the hollow transmission shaft 11 is four. These four limiting members 113 are correspondingly arranged in pairs on two opposite sides of the first end 111 of the hollow transmission shaft 11. The hollow transmission shaft 11 has a cylindrical shape. As such, along a circumferential direction of the hollow transmission shaft 11, a distance H between outer side walls 1131 of each pair of the limiting members 113 is greater than a width W of the latching protrusion 121 (as shown in FIG. 7). Since a U-shaped connecting member 31 of the pedicle screw assembly 3 has a U-shaped notch, a width of the U-shaped notch is slightly greater than the distance H, and the U-shaped connecting member 31 of the pedicle screw assembly 3 can abut against the outer side walls 1131 of the limiting members 113. Moreover, since the distance H is greater than the width W, the transmission connecting member 12 will not jam or block the pedicle screw assembly 3 during subsequent installation of the pedicle screw assembly 3, thereby enhancing assembly convenience and structural stability.

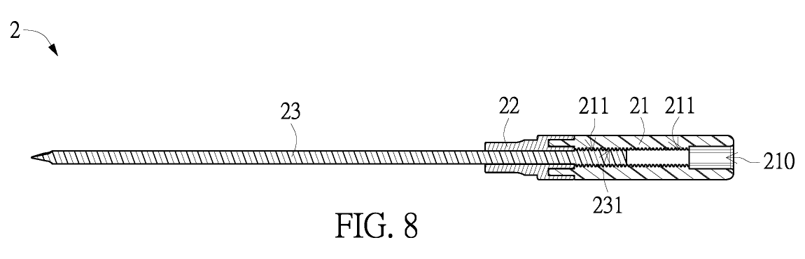

Reference is made to FIG. 8, which is a schematic cross-sectional view of the guide pin assembly of the screw implantation device according to the present disclosure. The guide pin assembly 2 includes a socket member 21, a coupling member 22, and a guide pin 23. The coupling member 22 is detachably connected to the socket member 21, and has a hollow structure. The guide pin 23 passes through the coupling member 22, and is connected to the socket member 21. For example, the socket member 21 has a shaft hole 210, and the shaft hole 210 is formed on a side opposite to the coupling member 22. An inner wall of the socket member 21 has an internal thread 211, a surface of the guide pin 23 has an external thread 231, and the socket member 21 is connected to the guide pin 23 through engagement between the internal thread 211 and the external thread 231. However, the present disclosure is not limited to the above-mentioned connection method of the socket member 21 and the guide pin 23.

In addition, the coupling member 22 includes two first protruding portions 221 and two second protruding portions 222. The two first protruding portions 221 and the two second protruding portions 222 are disposed on two sides of the coupling member 22. On any side of the coupling member 22, the second protruding portion 222 is adjacent to the first protruding portion 221, and there is a tiny gap between the second protruding portion 222 and the first protruding portion 221.

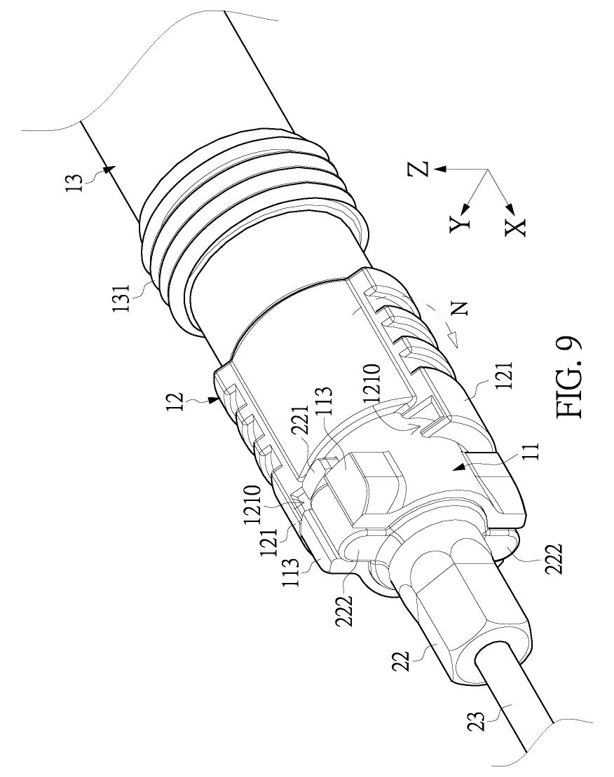

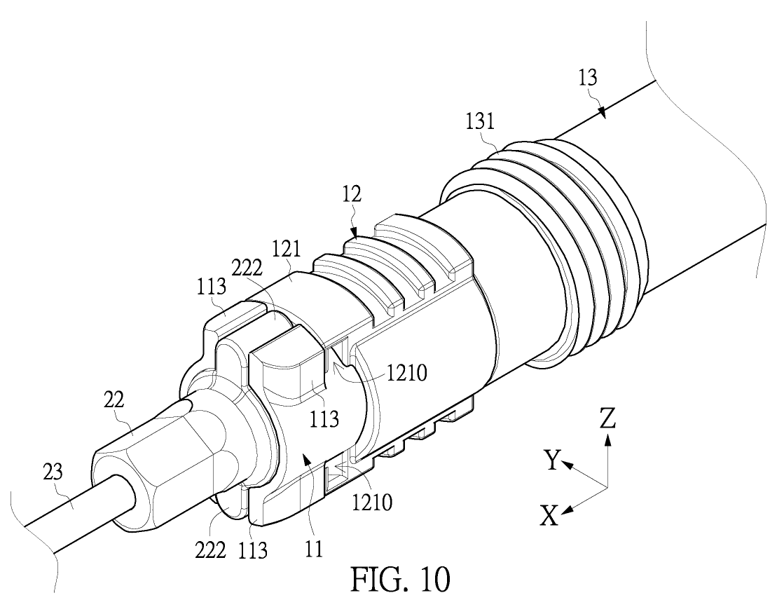

Referring to FIGS. 5, 9, and 10, FIGS. 9 and 10 are schematic views showing assembly of the transmission assembly and the guide pin assembly according to the present disclosure. The guide pin assembly 2 can be directly and detachably assembled to the first end 111 of the hollow transmission shaft 11. That is, the guide pin assembly 2 can be directly assembled to a front end of the screw implantation device D. In detail, when the guide pin assembly 2 is assembled to the hollow transmission shaft 11, the socket member 21 is inserted into the port 1111 of the first end 11, such that the hollow transmission shaft 11 is sleeved around the socket member 21 and connected to the coupling member 22. At this time, each of the second protruding portions 222 is engaged between a corresponding pair of the limiting members 113. Then, the transmission connecting member 12 is configured to rotate (which refers to a rotational direction N in FIG. 9) around an axis (which is parallel to an X-axis direction) of the hollow transmission shaft 11, such that the two first protruding portions 221 are respectively engaged in the two dents 1210.

Accordingly, the guide pin assembly 2 is fixedly connected to the transmission assembly 1, and high connection strength and stability therebetween can be ensured. Since each of the second protruding portions 222 is precisely engaged between a corresponding pair of the limiting members 113, any angular displacement of the guide pin assembly 2 after being assembled to the transmission assembly 1 can be effectively prevented, and the guide pin assembly 2 is ensured to synchronously rotate when being driven by the hollow transmission shaft 11 of the transmission assembly 1. Furthermore, since the two first protruding portions 221 are respectively blocked and limited by the two latching protrusions 121, unnecessary movement or sliding of the guide pin assembly 2 along an axial direction (the X-axis direction) can be effectively prevented, thereby enhancing stability and safety of the screw implantation device during an operational process. Through the design of latching and blockage, the guide pin assembly 2 is ensured to be tightly fixed and not loosened when a user operates the screw implantation device D, and an operational error that may occur due to a minute displacement can also be avoided, such that precision and reliability of the entire screw implantation device D can be guaranteed.

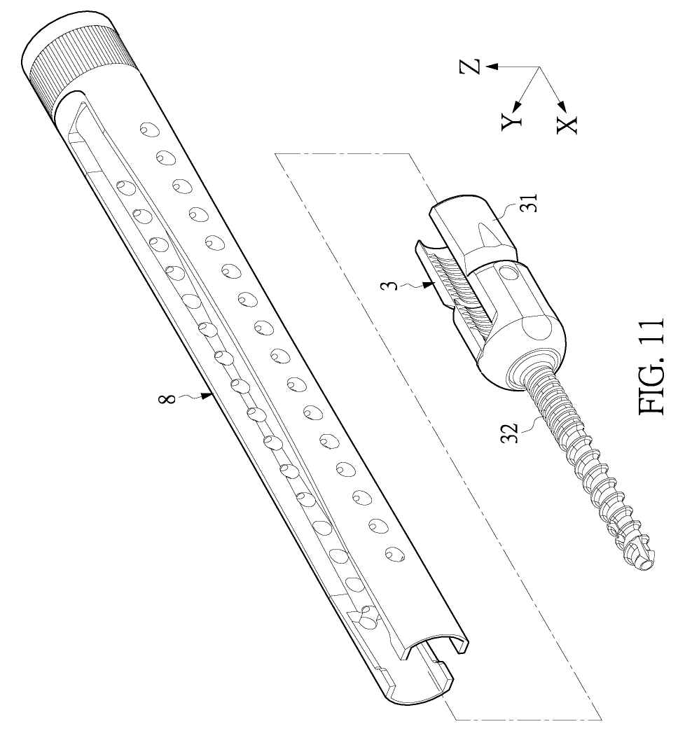

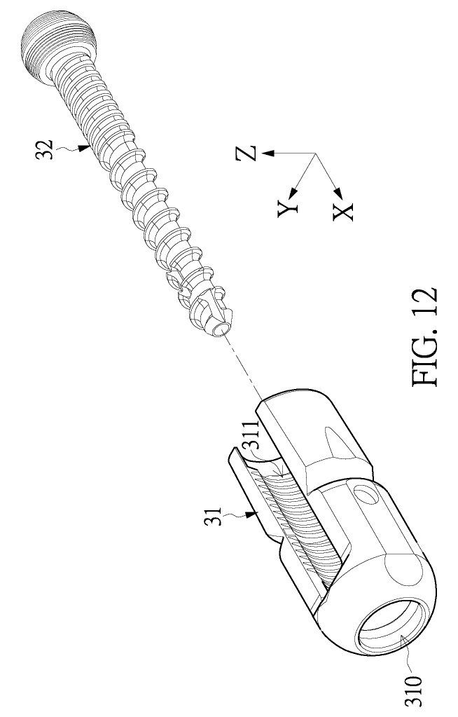

Referring to FIGS. 11 and 12, the pedicle screw assembly 3 includes a U-shaped connecting member 31 and a hollow pedicle screw 32. The U-shaped connecting member 31 has an opening 310, and the hollow pedicle screw 32 passes through the opening 310 and is engaged with the U-shaped connecting member 31. It should be noted that rotation of the hollow pedicle screw 32 is not limited by the U-shaped connecting member 31.

Referring to FIGS. 2, 3, 11, and 12, the pedicle screw assembly 3 is connected to the guide pin assembly 2, and the pedicle screw assembly 3 and the guide pin assembly 2 are jointly assembled to the first end 111 of the hollow transmission shaft 11. The retaining member 8 is sleeved around the transmission assembly 1, and is connected to the U-shaped connecting member 31. An inner wall of the U-shaped connecting member 31 has an internal thread 311, and an outer wall of the hollow connecting shaft 13 has an external thread 131. When the guide pin assembly 2 and the pedicle screw assembly 3 are assembled to the first end 111 of the hollow transmission shaft 11, the hollow connecting shaft 13 is connected to the U-shaped connecting member 31 through engagement between the internal thread 311 and the external thread 131, such that the hollow connecting shaft 13 is further connected to the pedicle screw assembly 3. At the same time, the guide pin 23 passes through the hollow pedicle screw 32, the coupling member 22 abuts against the hollow pedicle screw 32, and the hollow transmission shaft 11, the coupling member 22, and the hollow pedicle screw 32 are coaxially arranged.

As shown in FIG. 2, the central shaft 5 and the reducer assembly 6 are dynamically coupled to the driving motor 4. The driving motor 4 is configured to output power for driving the central shaft 5 to rotate along a rotational direction. The reducer assembly 6 is disposed between the driving motor 4 and the second end 112 of the hollow transmission shaft 11, and the reducer assembly 6 is configured to be connected to the driving motor 4 and the hollow transmission shaft 11. The quick-release connector 7 is connected to the reducer assembly 6. The quick-release connector 7 has an engaging groove 71. Through the engaging portion 1121 at the second end 112 of the hollow transmission shaft 11, the hollow transmission shaft 11 is engaged in the engaging groove 71 for axial positioning, so as to transmit a torque provided by the driving motor 4.

It should be noted that the second end 112 of the hollow transmission shaft 11 also has a port (not shown in the drawing). Hence, when the engaging portion 1121 is engaged in the engaging groove 71, the central shaft 5 is also inserted into the hollow transmission shaft 11 through said port, and a front end 51 of the central shaft 5 is inserted into the shaft hole 210 of the socket member 21. Accordingly, the socket member 21 is engaged with the central shaft 5, thereby enabling the central shaft 5, the socket member 21, and the guide pin 23 to be coaxially arranged (as shown in FIGS. 2 and 8). After the transmission assembly 1 is assembled with the driving motor 4, the central shaft 5, and the reducer assembly 6, the hollow transmission shaft 11 of the transmission assembly 1 is sleeved around and coaxially arranged with the central shaft 5, the driving motor 4 is adjacent to the second end 112 of the hollow transmission shaft 11, and the hollow transmission shaft 11 is dynamically coupled to the driving motor 4.

### Beneficial Effects of the Embodiment

In conclusion, in the screw implantation device D provided by the present disclosure, through the design of the first protruding portions 221 and the second protruding portions 222, the guide pin assembly 2 can be stably and reliably assembled to the hollow transmission shaft 11 of the transmission assembly 1. Since each of the second protruding portions 222 is engaged between a corresponding pair of the limiting members 113, any angular displacement of the guide pin assembly 2 after being assembled to the transmission assembly 1 can be effectively prevented, and the guide pin assembly 2 is ensured to synchronously rotate when being driven by the hollow transmission shaft 11 of the transmission assembly 1.

Furthermore, since the two first protruding portions 221 are respectively blocked and limited by the two latching protrusions 121, unnecessary movement or sliding of the guide pin assembly 2 along an axial direction (the X-axis direction) can be effectively prevented, thereby enhancing stability and safety of the screw implantation device during an operational process. Through the design of latching and blockage, the guide pin assembly 2 is ensured to be tightly fixed and not loosened when a user operates the screw implantation device D, and an operational error that may occur due to a minute displacement can also be avoided, such that precision and reliability of the entire screw implantation device D can be guaranteed.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A screw implantation device, comprising:
a transmission assembly, wherein the transmission assembly includes:
a hollow transmission shaft having a first end and a second end, wherein a plurality of limiting members are disposed at the first end; and
a transmission connecting member, wherein the transmission connecting member is rotatably sleeved around the first end, two latching protrusions are disposed on two sides of the transmission connecting member, and each of the latching protrusions has a dent; and
a guide pin assembly detachably assembled to the first end of the hollow transmission shaft, wherein the guide pin assembly includes:
a socket member;
a coupling member having a hollow structure, wherein the coupling member is detachably connected to the socket member, the hollow transmission shaft is sleeved around the socket member and connected to the coupling member, and two first protruding portions are disposed on two sides of the coupling member; and
a guide pin passing through the coupling member and connected to the socket member;
wherein, when the guide pin assembly is assembled to the hollow transmission shaft, the transmission connecting member is configured to rotate around an axis of the hollow transmission shaft, such that the two first protruding portions are respectively engaged in the two dents and blocked by the two latching protrusions, so as to fix the guide pin assembly in place.

2. The screw implantation device according to claim 1, wherein a quantity of the plurality of limiting members is four, the four limiting members are correspondingly arranged in pairs on two opposite sides of the first end and are arranged along a circumferential direction, and a distance between outer side walls of each pair of the limiting members is greater than a width of the latching protrusion.

3. The screw implantation device according to claim 2, wherein two second protruding portions are further disposed on the two sides of the coupling member, and the two second protruding portions are respectively adjacent to the two first protruding portions; and wherein, when the guide pin assembly is assembled to the hollow transmission shaft, each of the two second protruding portions is engaged between a corresponding pair of the limiting members.

4. The screw implantation device according to claim 1, further comprising a pedicle screw assembly, wherein the pedicle screw assembly includes a U-shaped connecting member, the transmission assembly further includes a hollow connecting shaft, the hollow connecting shaft is sleeved around the hollow transmission shaft, and the transmission connecting member is disposed between the plurality of limiting members and the hollow connecting shaft; wherein an outer wall of the hollow connecting shaft has an external thread, and an inner wall of the U-shaped connecting member has an internal thread; and wherein, when the pedicle screw assembly is assembled to the first end of the hollow transmission shaft, the hollow connecting shaft is connected to the U-shaped connecting member through engagement between the internal thread and the external thread.

5. The screw implantation device according to claim 4, further comprising:
a driving motor;
a central shaft dynamically coupled to the driving motor, wherein the hollow transmission shaft of the transmission assembly is sleeved around and coaxially arranged with the central shaft, the driving motor is adjacent to the second end of the hollow transmission shaft, and the hollow transmission shaft is dynamically coupled to the driving motor;
a reducer assembly disposed between the driving motor and the second end of the hollow transmission shaft, wherein the reducer assembly is configured to be connected to the driving motor and the hollow transmission shaft; and
a quick-release connector connected to the reducer assembly, wherein the quick-release connector has an engaging groove, an engaging portion is disposed at the second end of the hollow transmission shaft, and the engaging portion is engaged in the engaging groove, so as to axially position the hollow transmission shaft and transmit a torque provided by the driving motor.

6. The screw implantation device according to claim 5, wherein the pedicle screw assembly is detachably assembled to the first end of the hollow transmission shaft and connected to the guide pin assembly; and wherein the pedicle screw assembly includes a hollow pedicle screw, and the hollow transmission shaft, the coupling member, and the hollow pedicle screw are coaxially arranged.

7. The screw implantation device according to claim 6, wherein the U-shaped connecting member has an opening, and the hollow pedicle screw passes through the opening and is engaged with the U-shaped connecting member.

8. The screw implantation device according to claim 7, wherein, when the guide pin assembly and the pedicle screw assembly are assembled to the first end of the hollow transmission shaft, the guide pin passes through the hollow pedicle screw, and the coupling member abuts against the hollow pedicle screw.

9. The screw implantation device according to claim 7, further comprising a retaining member, wherein the retaining member is sleeved around the transmission assembly, and is connected to the U-shaped connecting member.

10. The screw implantation device according to claim 5, wherein the central shaft, the socket member, and the guide pin are coaxially arranged, and the socket member is engaged with the central shaft.

11. The screw implantation device according to claim 1, wherein an inner wall of the socket member has an internal thread, a surface of the guide pin has an external thread, and the socket member is connected to the guide pin through engagement between the internal thread and the external thread.
